(19) United States
(12) Patent Application Publication
(10) Pub. No.: US 2026/0232888 A1
(43) Pub. Date: Aug. 13, 2026

(54) MODULAR BIOLOGICAL CELL PROCESSING SYSTEM

(71) Applicant: Fenwal, Inc., Lake Zurich, IL (US)

(72) Inventors: Christopher J. WEGENER, Libertyville, IL (US); James G. MADSEN, Barrington, IL (US); Kyle THOMPSON, Vernon Hills, IL (US); Alexander DODGE, Lincolnshire, IL (US); Paige BOTHWELL, Palatine, IL (US)

(21) Appl. No.: 19/155,530

(22) PCT Filed: Feb. 12, 2024
(86) PCT No.: PCT/US2024/015424

§ 371 Date: Aug. 11, 2025

Related U.S. Application Data
(60) Provisional application No. 63/484,636, filed on Feb. 13, 2023.

(51) Int. Cl. A61M 1/36; A61M 1/02; A61M 60/113; A61M 60/258; A61M 60/39; A61M 60/427; A61M 60/508; A61M 60/851

(52) U.S. Cl. CPC A61M 1/3693; A61M 1/0209; A61M 1/36225; A61M 60/113; A61M 60/258; A61M 60/39; A61M 60/427; A61M 60/508; A61M 60/851

(57) ABSTRACT

Systems are provided for collecting, sorting, and concentrating blood components from whole blood for reinfusion. The system may further include cell modification. The system includes a blood processing device and a fluid flow circuit.

### CROSS-REFERENCE TO RELATED APPLICATIONS

This application claims the benefit of and priority to U.S. Provisional Patent Application Ser. No. 63/484,636, filed Feb. 13, 2023, the contents of which are incorporated by reference herein.

### BACKGROUND

### Field of the Disclosure

The present disclosure relates to biological cell, including blood and blood component, processing. More particularly, the present disclosure relates to systems and methods for collecting biological cells (e.g., blood components) and processing/modifying the cells before reinfusion into the patient.

### Description of Related Art

Various blood processing systems make it possible to separate blood into two or more constituent parts, which may be useful for donation purposes and for treatment of individuals with potentially detrimental or harmful conditions or disorders.

When such systems are used for blood component donation, whole blood is typically drawn from a donor, a particular blood component or constituent is removed and collected, and the remaining blood constituents are returned to the donor.

Such systems can also be used to provide blood components for use in cellular therapies for patients. For these therapies it is typical to separate a particular cellular or other blood component from whole blood and modify, enrich and/or expand the collected component before returning it to the patient as part of a therapeutic treatment. For example, one such therapy, Chimeric antigen receptor (CAR) T-cell therapy, alters a patient's T-cells and adds an artificial receptor to the cells that attach to cancer cell antigens. These modified T-cells are returned to the patient and can help target and destroy specific cancer cells.

These modified therapeutic cells are typically produced in manufacturing facilities separate from the blood collection site. This manufacturing process can be lengthy and onerous and result in a substantial gap between the time the cells are collected, modified or otherwise treated and then are reinfused to the patient as part of a therapy.

Therapies, such as CAR-T therapy show incredible efficacy for hematological malignancies in the clinic, however, many patients are rendered ineligible for treatment because the vein-to-vein times for administering these autologous gene-modified therapies are too long. There is, therefore, a need for rapid manufacture and reinfusion of therapeutic cells such as, but not limited to, gene modified autologous cells.

It would, therefore, be desirable to provide an integrated system which can collect the cells, concentrate the cells, modify the cells, and prepare the cells for reinfusion and/or reinfuse the cells efficiently and without substantial delay.

### SUMMARY

There are several aspects of the present subject matter which may be embodied separately or together in the devices, systems and methods described and claimed below. These aspects may be employed alone or in combination with other aspects of the subject matter described herein, and the description of these aspects together is not intended to preclude the use of these aspects separately or the claiming of such aspects separately or in different combinations as set forth in the claims appended hereto.

In one aspect, a fluid flow circuit for use in a blood processing system includes a microfluidic sorter module, a cell concentrator module, at least one pump, at least one fluid reservoir for holding fluids during the blood processing, a valve system, at least one fluid source container, at least one cell modification module, a blood source access device; and a plurality of conduits fluidly connecting the components of the fluid flow circuit.

### BRIEF DESCRIPTION OF THE DRAWINGS

FIG. 1 is a schematic view of an exemplary fluid flow circuit;

FIG. 2 is a schematic view of a blood processing device;

FIG. 3A is a perspective view of an exemplary fluid flow circuit;

FIG. 3B is a perspective view of an open blood processing device with an inserted fluid flow circuit;

FIG. 3C is a perspective view of a blood processing system;

FIG. 4 is a front perspective view of an exemplary fluid flow circuit;

FIG. 5 is a rear perspective view of an exemplary fluid flow circuit;

FIG. 6 is a schematic view of an exemplary first portion of a disposable fluid flow circuit;

FIG. 7 is a schematic view of another exemplary first portion of a disposable fluid flow circuit;

FIG. 8 is a schematic view of an exemplary second portion of a disposable fluid flow circuit;

FIG. 9 is a schematic view of a priming step in the disposable fluid flow circuit of FIG. 8;

FIG. 10 is a schematic view of a first separation step in the disposable fluid flow circuit of FIG. 8;

FIG. 11 is a schematic view of a second separation step in the disposable fluid flow circuit of FIG. 8;

FIG. 12 is a schematic view of a cell concentration step in the disposable fluid flow circuit of FIG. 8;

FIG. 13 is a schematic view of a first cell formulation step in the disposable fluid flow circuit of FIG. 8;

FIG. 14 is a schematic view of a second cell formulation step in the disposable fluid flow circuit of FIG. 8;

FIG. 15 is a schematic view of a gene delivery step in the disposable fluid flow circuit of FIG. 8;

FIG. 16 is a schematic view of a cell selection step in the disposable fluid flow circuit of FIG. 8;

FIG. 17 is a schematic view of one embodiment of the biological cell processing system;

FIG. 18 is a schematic view of one embodiment of the biological cell processing system;

FIG. 19 is a schematic view of one embodiment of the biological cell processing system;

FIG. 20 is a schematic view of a pressure control system of one embodiment of the biological cell processing system;

FIG. 21 is a schematic view of a fluid flow path of one embodiment of the biological cell processing system; and

FIG. 22 is a schematic view of a fluid flow path of one embodiment of the biological cell processing system.

### DESCRIPTION OF THE ILLUSTRATED EMBODIMENTS

The embodiments disclosed herein are for the purpose of providing a description of the present subject matter, and it will be understood that the subject matter may be embodied in various other forms and combinations not shown in detail. Therefore, specific designs and features disclosed herein are not to be interpreted as limiting the subject matter as defined in the accompanying claims.

The disclosure will be more fully understood from the following description taken in conjunction with the accompanying drawings. Some of the figures may have been simplified for the purpose of more clearly showing other elements. Such simplification of the figures are not necessarily indicative of the presence or absence of particular elements in any of the exemplary embodiments, except as may be explicitly delineated in the corresponding written description. The drawings are not necessarily to scale.

The current disclosure includes exemplary embodiments of fluid flow circuits and blood processing devices, which are combinable to form an automated blood processing system for collecting, separating, concentrating, and modifying blood cells for reinfusion back into a patient.

“Blood” includes without limitation blood and blood components, and “cell” or “biological cell” includes without limitation blood cells, such as red cells, white blood cells, and T-cells. By “automated,” it is meant that the apparatus can be programmed to carry out the processing steps of a biological fluid processing method without substantial operator involvement. Of course, even in the automated system of the present disclosure, it will be understood that some operator activity may be involved, including the loading of the disposable fluid circuits and entering processing parameters. Additional manual steps may be required as well. However, the reusable apparatus can process blood through the disposable circuit(s) described below without substantial operator intervention.

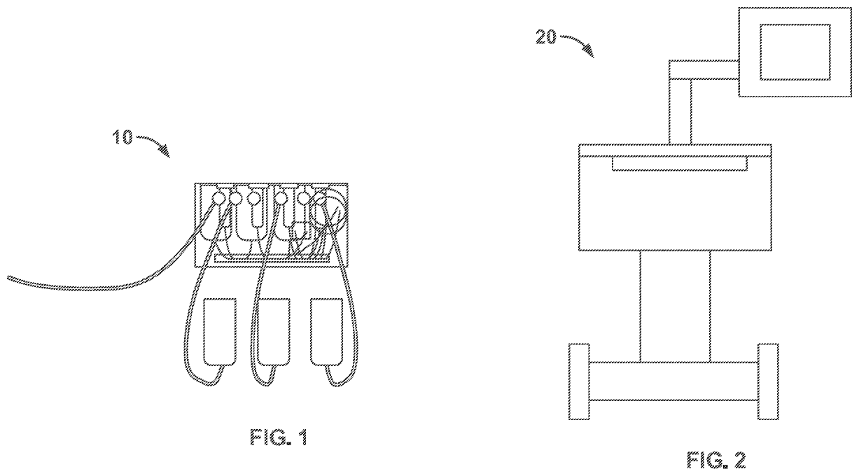

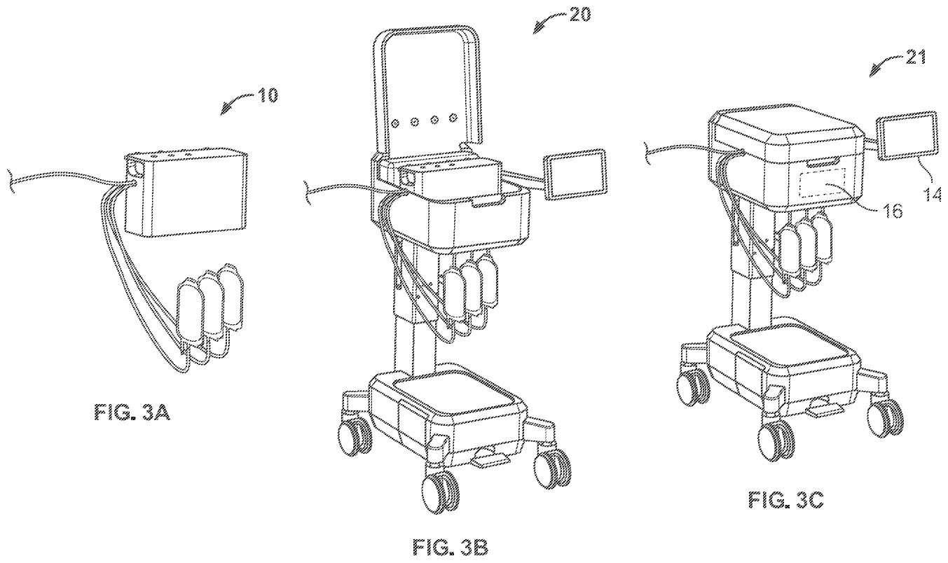

As illustrated in FIGS. 1 and 2, the blood processing system includes two principal components, a durable and reusable blood or cell processing device 20 (FIG. 2) and a disposable fluid flow circuit (FIG. 1)(collectively referenced herein as element 10). The blood processing device includes components that control and monitor fluid flow through the disposable flow circuit 10, and a controller 16 (FIG. 3C), which governs and/or directs the operation of the other components of the blood processing device 20 to perform a blood processing procedure selected by the operator, as will be described in greater detail.

Blood processing systems and methods according to the current disclosure are described as utilizing a blood processing device or system and different cell separating, concentrating and modifying modules. However, it should be understood that the principles described herein are not limited to a particularly configured device and/or a particular sequence of steps or stages. Rather, the blood processing systems and methods described herein may be applied using a variety of differently configured blood processing devices and fluid flow circuits that carry out blood processing procedures in different ways.

The blood processing device 20, as shown in FIGS. 2, 3B and 3C, can be modular and designed with a plurality of components that work with various fluid flow circuits 10 to process blood components. The device 20 may include movable or mobile components, such as wheels, for moving the device to and from a patient's bed or chair. The device may be placed at least substantially or even completely at the patient point-of-care i.e., bedside. The device can accommodate all manufacturing unit operations in a single system, as will be described below. The blood processing device may include valves or valve components, air control systems, pumps, detectors, sensors, a controllers, a user interface, and any other components that may function with to aid with moving fluid through the fluid flow circuit 10.

Blood processing device 20 may include valves or motors associated with valve portions of the fluid flow circuit 10. The valves may be configured to interact with the conduits of a fluid flow circuit 10 mounted to the device 20. As examples only, the valves can be solenoid pinch valves, motor-driven rotary pinch valves, linear actuators, stop cocks or any other type of automated clamping or valve device known in the art. In an exemplary embodiment, the blood processing device 20 includes valve motors compatible with valve components on the fluid flow circuit 10.

The blood processing device 20 may also include air control systems for providing air to, for example, pneumatic syringe pump assemblies, discussed further below. The air control system may include a vacuum and/or pressure source, such as a diaphragm pump. The vacuum or pressure source may pump filtered air in and out of the pump. Examples and further details of assemblies that may be utilized in the blood processing device 20 are described in U.S. patent Publication Ser. Nos. 10,926,895, 11,191,880, 10,781,001 and U.S. patent application Ser. No. 17/026,156.

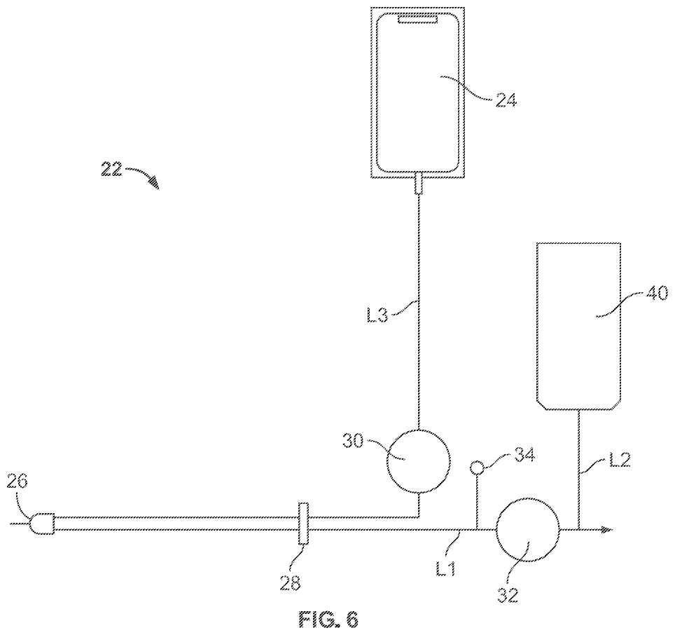

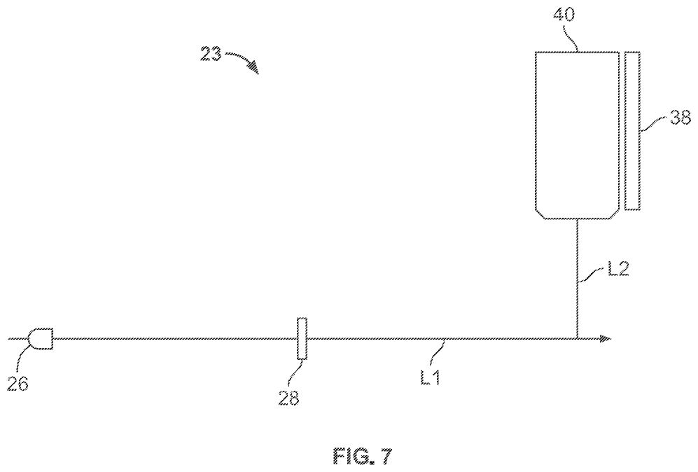

The blood processing device 20 may also include a plurality of pumps as part of the user adjacent portion (by way of example, FIG. 6 includes two possible pumps 30,32) to initiate and cause fluid to flow through the fluid flow circuit 10. If a user adjacent portion is not utilized or a passive user adjacent portion is utilized, such as shown in FIG. 7, the blood processing device does not need to include these pumps. The pumps may be differently or similarly configured and/or function similarly or differently from each other. In an embodiment, the pumps are configured as peristaltic pumps, which may be generally configured as described in U.S. Pat. No. 5,868,696. Each pump may engage a different line and may be selectively operated under command of the controller 16 to cause fluid to flow through a portion of the fluid flow circuit 10, when using a user adjacent portion of the fluid flow circuit.

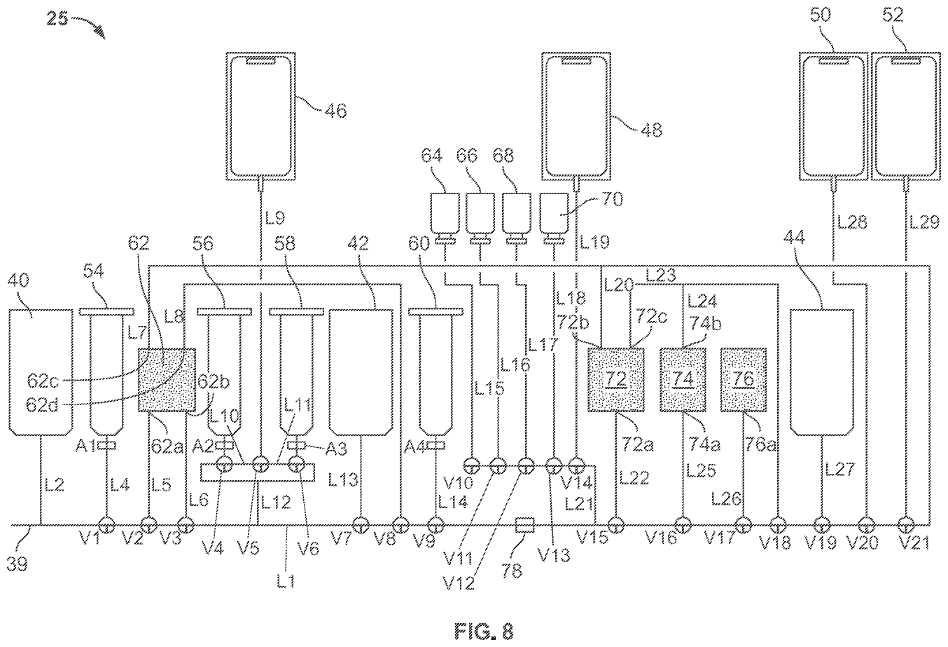

The illustrated blood processing device 20 can also include air detectors, labeled “A” on the schematic of the fluid flow circuit in FIG. 8, (e.g., an ultrasonic bubble detector), which accommodates tubing of the fluid flow circuit 10 that flows fluid to a recipient. It may be advantageous to prevent air from reaching the recipient, whether a human recipient (e.g., the same human that serves as the blood source) or a non-human recipient (e.g., a storage bag or container), so the air detectors may transmit signals to the controller 16 that are indicative of the presence or absence of air in the tubing. If the signal is indicative of air being present in the tubing, the controller 16 may initiate an alarm or error condition to alert an operator to the condition and/or to take corrective action to prevent the air from reaching the recipient (e.g., by reversing the flow of fluid through the tubing or diverting flow to a vent. The air detectors may alternatively or additionally be used as part of a fluid flow control for the fluid flow circuit 10.

The illustrated blood processing device 20 may also include one or more sensors or sensing elements for sensing a condition or characteristic of a blood component. For example, in one embodiment, a cell density sensor 78 (such as shown in FIGS. 8-16 on line L1) may be incorporated to detect absolute or relative changes in cell concentration. Optical devices that use methods such as light transmission, scatter, or spectroscopy could be used. Devices that utilize electrical methods such as capacitance could also be useful. Even devices that use acoustic methods could yield relational density measurements.

Also, the plunger position of any of the syringe pumps 54, 56, 58, 60 of the fluid flow circuit 10 (shown in FIG. 8 and described below) may be tracked by a sensing element. Additionally, pressure sensors may be incorporated into the system to monitor the pressure at various locations of the fluid flow circuit 10. For instance, if the blood source is a human donor, one or more pressure sensors, such as donor pressure sensor 34 (FIG. 6) may be configured to monitor the pressure of the donor's vein during blood draw and return. The controller 16 may receive signals from the pressure sensor that are indicative of the pressure within the fluid flow circuit 10 and, if a signal indicates a low-or high-pressure condition, the controller 16 may initiate an alarm or error condition to alert an operator to the condition and/or to attempt to bring the pressure to an acceptable level without operator intervention.

As noted above, the blood processing device 20 includes a controller, such as the controller 16 shown in FIG. 3C. Although shown in the upper part of the blood processing device 20, the controller may be incorporated into different parts of the blood processing device 20. The controller may, according to the embodiments described herein, include a programmable microprocessor, which microprocessor may be programmed to operate the blood processing device 20 and system 21 according to a process.

According to other embodiments, the controller may include one or more electrical circuits designed to carry out the actions described herein. In addition, the controller may include one or more memories. The instructions by which the microprocessor is programmed may be stored on the memory associated with the microprocessor, which memory/memories may include one or more tangible non-transitory computer readable memories, having computer executable instructions stored thereon, which when executed by the microprocessor, may cause the microprocessors to carry out one or more actions as described below.

The controller may be coupled to one or more of the structures of the blood processing device 20 and also structures of the fluid flow circuit 10 (FIG. 8), for example to receive information (e.g., in the form of signals) from these structures or to provide commands (e.g., in the form of signals) to these structures to control the operation of the structures. The controller may be coupled to the sensors, valves, and pumps to provide commands to those devices to control their operation. It may also be possible that the controller receives information from and provides commands to a given structure, such as one of the structures already mentioned. The controller may be directly electrically connected to these structures to be coupled to them, or the controller may be directly connected to other intermediate equipment that is directly connected to these structures to be coupled to them.

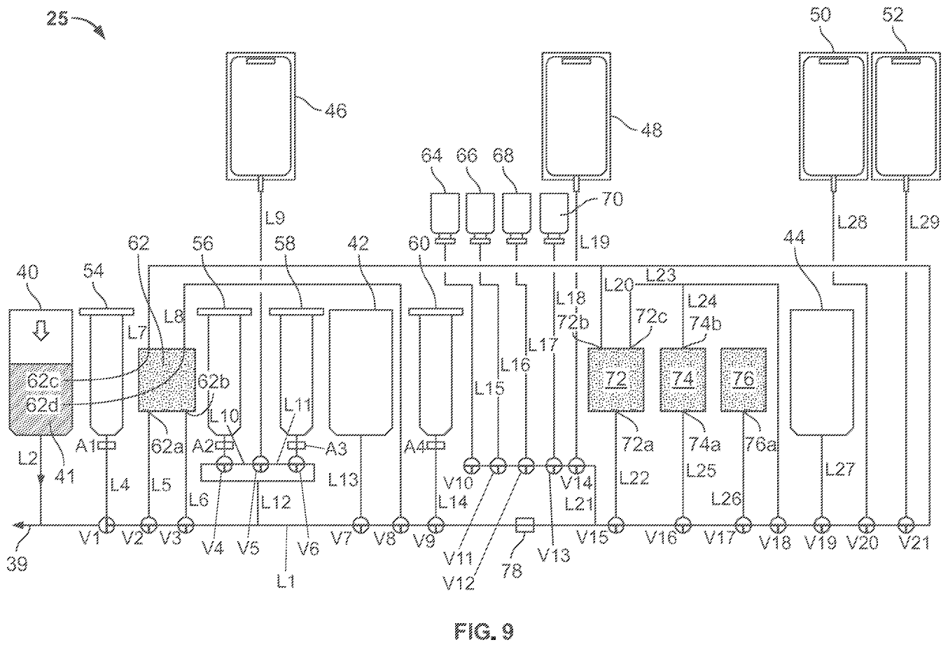

The controller is configured and/or programmed to execute at least one blood processing procedure (such as shown in FIGS. 9-16) but, more advantageously, is configured and/or programmed to execute multiple types of blood processing procedures which may include a separating portion and a cell modifying portion.

More particularly, in carrying out any blood processing procedure, the controller is configured and/or programmed to control the flow of fluid and amount of a fluid from one component to another. This may include instructing the valves to open and close at specific points during a procedure or initiating transfer of a fluid from one container to another. Hence, while it may be described herein that a particular component of the blood processing system performs a particular function, it should be understood that that component is being controlled by the controller to initiate and/or perform that function.

A user interface screen 14 (e.g., a touchscreen) may be associated with the blood processing device 20 (as in FIG. 3C). The user interface screen 14 may allow an operator to interact with the system controller (e.g., a microprocessor) of the device 20 to provide instructions to the controller (e.g., to carry out a particular procedure), as well as providing information to the controller to be used during a procedure (e.g., white blood cell (WBC) pre-count of the blood of the blood source). The user interface screen 14 may serve as a display to provide the operator with instructions (e.g., to connect or disconnect the blood source from the flow circuit 10) and information (e.g., alerting the operator to a blockage in a fluid flow conduit of the flow circuit 10) and providing a procedure status.

The blood processing device 20 may include computer equipment that permits the blood processing device 20 including the controller 16 to communicate (whether via wires, cables, etc. or wirelessly) with other blood processing devices over a local network, or with other blood processing devices or other computer equipment (e.g., a server) over local networks, wide area networks, or the Internet. According to such an embodiment, the device may include an internal transmitter/receiver device.

FIGS. 3A-3C show insertion and loading steps of a fluid flow circuit 10 into the blood processing device 20 to create single, modular blood processing system 21. The fluid flow circuit 10 (FIG. 3A) is inserted into an open cabinet of the blood processing device (FIG. 3B) and the components are connected as needed before closing the cabinet in a third step (FIG. 3C). Blood processing system 21, including the blood processing device 20 and fluid flow circuit is a single modular system, which is capable of performing the entirety of the blood processing procedure without movement to outside components or devices.

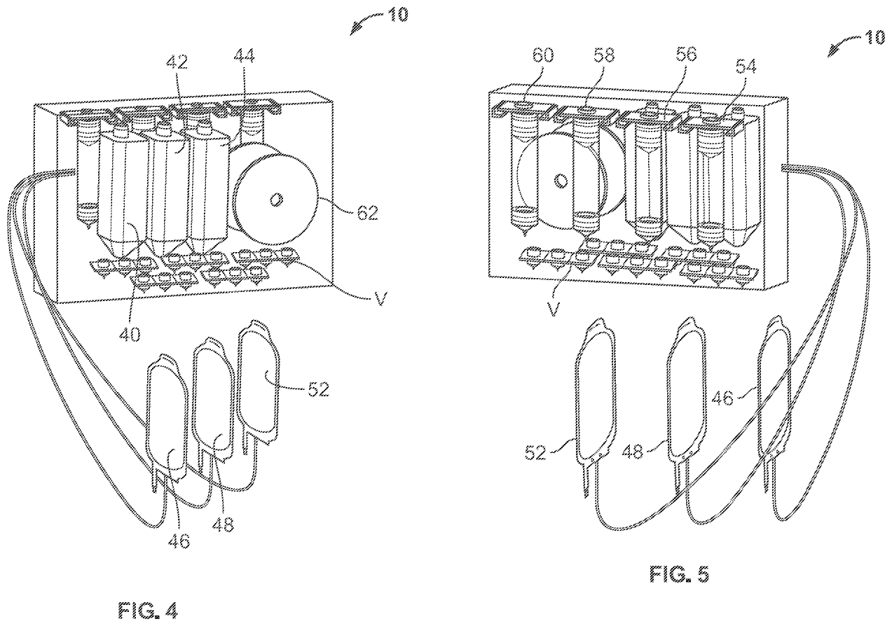

As for the fluid flow circuit or flow set 10 (details in FIGS. 4-8), it is intended to be a sterile, single use, disposable item. FIGS. 4 and 5 include perspective views of the fluid flow circuit 10, and FIG. 8 is a schematic illustration of the fluid flow circuit. FIGS. 9-16 show different stages of an exemplary procedure. The fluid flow circuit is modular and different customizable configurations of the fluid flow circuit can be loaded into a blood processing device 20. Before beginning a given blood processing and modifying procedure, the operator loads the fluid flow circuit 10 into the blood (cell) processing device 20. The controller 16 implements the procedure based upon preset protocols, taking into account other input from the operator. Upon completing the procedure, the operator removes the fluid flow circuit 10 from association with the blood processing device 20. If any portions of the fluid flow circuit 10 are holding the components (such as doses), these are removed from the device 20 and retained for storage, transfusion, or further processing. The remainder of the fluid flow circuit 10 is removed from the blood processing device 20 and discarded.

The different fluid flow circuits used in combination with the blood processing device may vary slightly in components depending on the blood or cell processing procedure and resulting cells to be carried out using the system. Accordingly, different fluid flow circuits may be used in connection with particular blood processing procedures. The fluid circuit is completely customizable, and as such, various components can be added and removed. Generally speaking, the fluid flow circuit 10 may include pumps, reservoirs, valve components, fluid input and output containers, a separation device, a concentration device, and at least one cell modifying module as shown in FIG. 8 or combinations thereof.

The fluid flow circuit may include two different portions, a first user adjacent portion (22 or 23, shown in FIGS. 6 and 7), used when connecting the system to a donor and a second processing portion 25 (shown in FIG. 8). The user adjacent portion can be selected based on whether the process will be active (reinfusion back to the donor) or passive (dose collected for later infusion). FIG. 6 shows a first possible user adjacent portion 22 with components and a schematic of an active process. FIG. 7 shows a second possible user adjacent portion 23 with components and a schematic of a passive process.

Both user adjacent portions include at least one blood source access device 26 (e.g., phlebotomy needles), used to both draw blood from a blood source and convey fluid to the blood source. Optionally, two blood source access devices (e.g., dual needle) may be used with one serving to draw blood into the flow circuit 10 from a source and an additional may be used to return fluid to the source. In another embodiment, a blood source (such as a previously collected bag, may be attached to the system). Both user adjacent portions also include a donor isolation clamp 28. Main line L1 is present in both portions as well, connecting the fluid processing portion 25 (FIG. 8) to the user adjacent portion of the fluid flow circuit 10.

In the active user adjacent portion 22 of the fluid flow circuit 10, shown in FIG. 6, two separate flow lines connect to the blood source access device 26, a first line L3 connecting an anticoagulant container 24 and a second line L2 connecting to reservoir 40. The blood processing device 20 may include an associated anticoagulant pump 30 and a draw/return pump 32 for moving fluid to and from a patient.

The passive user adjacent portion 23, shown in FIG. 7, only requires connection to the reservoir 40 and optionally a level-sensing element 38 and therefore only includes a line L1. The anticoagulant may be added to reservoir 40 before processing.

The passive or active user adjacent portions may also include a plurality of pumps (FIG. 6 includes two possible pumps 30,32) to cause fluid to flow through the fluid flow circuit 10. The pumps may be differently or similarly configured and/or function similarly or differently from each other. In an embodiment, the pumps are configured as peristaltic pumps, which may be generally configured as described in U.S. Pat. No. 5,868,696. Each pump may engage a different line and may be selectively operated under command of the controller 16 to cause fluid to flow through a portion of the fluid flow circuit 10.

Turning now to the fluid processing portion 25, a possible fluid processing portion 25 of the fluid flow circuit 10 is shown in FIG. 8. The fluid flow circuit 10 may include a plurality of fluid input and output containers. Each container may be integrally formed with the fluid flow circuit 10 or they may be connected to the fluid flow circuit (e.g., by piercing a septum of a tube of the fluid flow circuit, via a luer connector, or by sterilely joining using a sterile welding system) before the fluid flow circuit is connected to the blood processing device, forming the blood processing system 21. The containers may be composed of any desired medical grade material, such as medical grade plastic. The fluid input containers included on the processing portion 25 of the fluid flow circuit may include at least one buffer container 46 and at least one solution or liquid container 64. The solution or liquid containers may be configured to hold a liquid chemical composition for mixing with blood or blood cell components. FIG. 8 shows optional first buffer container 46 and second buffer container 48 and optionally four solution or liquid containers 64, 66, 68, 70. However, the amount of buffer containers and the amount and/or presence of solution or liquid containers may vary based on the cell modification process being utilized.

Output containers may also be integrally formed with the fluid flow circuit 10 or may be connected to the fluid flow circuit 10. These containers are meant to house different cellular fractions, spent buffers, formulated suspensions, or accommodate samples thereof. These containers may include a waste container 52 or final dose container 50 also shown in FIG. 8. In a process where the dose is not immediately returned to the patient after modification (and removed from the system) the dose container 50 may be a removable container which is transferred after the procedure.

The fluid flow circuit may include valves or valve arrays (V1-V21 as shown in FIGS. 8-16). The valves may be stopcock valves. These valves cooperate with/interface with motors that are part of the blood processing device hardware. The valves may be used to direct flow between different elements of the fluid flow circuit. Other types of valves, such as solenoid-driven valves are described below.

The fluid flow circuit may also include pumps 54,56,58,60. Although four pumps are shown in FIG. 8, the fluid flow circuit may utilize more or fewer pumps. The pumps are preferably pneumatic syringe pump assemblies that interface with air controls systems on the blood processing device 20 hardware. Positive or negative pressure can be applied to displace the syringe plunger. Positive pressure translates to fluid flow out of the pump, negative into the pump. Optionally, there is a sterile filter embedded with a cap of the syringe. In an embodiment, the pumps may be generally configured as described in U.S. Patent Application No. 2021/0121827, which is hereby incorporated by reference in its entirety. The plunger position may be tracked by a sensing element on the hardware. Pumps can be operated in pressure-targeting mode or in flow-rate targeting mode, depending on the control scheme required for that processing step. The pumps may also be configured as pneumatic syringe pumps as described in U.S. Application No. 63/615,004, filed Dec. 27, 2023, and incorporated herein by reference.

The fluid flow circuit may include a plurality of reservoirs 40, 42, 44. Reservoirs are used as passive vessels to hold fluids before, during, and after processing steps. Reservoirs may be vented with sterilizing filters such that flow into or out of the reservoir does not result in pressurization of the vessel. Although FIG. 8 shows a fluid flow circuit with three reservoirs, more or less may be included in the fluid flow circuit 10.

The reusable hardware processing device may include at least one weigh scale associated with at least one of the containers of the fluid flow circuit. There may be a weigh scale associated with a first buffer container 46, a first buffer container 48, one or more of the four solution or liquid containers 64, 66, 68, 70, any of the reservoirs 40, 42, 44, the dose or sample container 50, and the waste container 52. Any of the containers of the circuit configured to hold fluid for a time may include a weigh scale to monitor the amount of liquid added or removed.

The fluid flow circuit 10 may include a separation module 62 (FIG. 8). As described in more detail below, a microfluidic separation module 62 may be employed to continuously separate particles or cells. The microfluidic separation module may include a plurality of channels used for separating by cell characteristic, such as diameter. For instance, the critical diameter of ~7 um could separate nucleated white blood cells from red blood cells and platelets. The separator module 62, as shown in FIG. 8 shows two outputs 62c and 62d. A first output 62d for cells larger than a designated critical diameter, and a second output 62c for cells smaller than a designated critical diameter. These separator modules can also be used to displace target cell populations into new buffers, effectively “washing” said cell suspensions. In alternate embodiments, the separator module may include a spinning membrane separator or centrifugal separation chamber utilized in other blood processing devices, such as those described in greater detail in U.S. Pat. No. 4,526,515 to DeVries; U.S. Pat. No. 5,194,145 to Schoendorfer; U.S. Pat. No. 6,312,607 to Brown et al. ; U.S. Pat. No. 6,524,231 to Westberg et al.; U.S. Pat. No. 4,094,461 to Kellogg et al. ; U.S. Pat. No. 7,052,606 to Gibbs et al.; U.S. Pat. No. 4,300,717 to Latham; U.S. Pat. No. 8,075,468; and U.S. Patent Application Publication No. 2009/0215602 to Min et al., all of which are hereby incorporated by reference. If a spinning membrane separator or centrifugal separator are utilized, the blood processing device may include the applicable hardware components.

The fluid flow circuit 10 may include a concentrator module or microfluidic concentrator module 72 may be employed to continuously concentrate particles or cells, generating a concentrated output stream at outlet 72c and a supernatant output stream at outlet 72b from a single input stream at inlet 72a as shown in FIG. 8. The concentrator module 72 may operate at a fixed concentration rate (for instance 10×) per pass through the module. Desired cell concentrations can be targeted by performing series of fixed concentrations and dilution steps. In one example, white blood cells may be concentrated 25×. Alternately, the concentrator module may achieve a variable concentration by passing through the module multiple times and including a dilution between passes. As an example, a concentration such as 15× can be achieved by this method.

The fluid flow circuit 10 may be further configured to interface with or include at least one cell modification module. In one example, these modules can perform cell and/or gene therapy. Accordingly, the fluid flow circuit 10 may be configured to interface with one or more of a gene delivery module and a cell selection module.

A gene-delivery module 74 may also be part of the fluid flow circuit 10 as shown in FIG. 8. The module may be employed to administer intracellular payloads and cell suspension. The gene delivery module can include an inlet 74a and an outlet 74b. Examples of modules may be electroporators, mechanoporators, sonoporators, soluporators or other flow through transfection technologies and/or means of introducing therapeutic payloads. The gene-delivery module may include the electroporation device disclosed in U.S. Patent Publication No. 2020/0282116.

A cell selection module 76, such as an affinity-based cell selection module may also be part of the fluid flow circuit 10 as shown in FIG. 8. The cell selection module 76 may be employed to phenotypically isolate a target cell population from a bulk cell suspension. The cell selection module 76 may include an inlet 76a which may also function as an outlet. Technologies such as magnetic-beads, affinity chromatography, or filtration could be implemented using positive or negative selection methods. The cell selection module may include the affinity column as described in U.S. Patent Application No. 63/613,500, filed Dec. 21, 2023.

Alternatively, the fluid flow circuit 10 may utilize existing portions of the fluid flow circuit 10 as a cell modification module such as for cell formulation. In these instances, a solution or liquid chemical additive may be added to blood cells in a reservoir that is integrated with the fluid flow circuit (such as 42,44) or pump (such as 60) or both. The blood cells may formulate, mix, or incubate in this reservoir or be moved between components of the fluid flow circuit 10.

As noted, the various components of the fluid flow circuit 10 may be connected by flexible tubing or any other suitable fluid flow conduit. In one embodiment, the fluid flow circuit may include a cassette. The cassette may be rigid or flexible. The fluid flow circuit 10 includes lines L1-L32 (shown in detail in FIG. 8), and more or less lines may be added or subtracted depending on the desired configuration of the fluid flow circuit 10 and the attached modules.

Various additional components may be incorporated into the fluid flow circuit. For example, a return line filter may be associated with a line leading to a fluid recipient, filters may be positioned upstream of one or more of the fluid containers to remove a substance (e.g., leukocytes) from a separated component (e.g., red blood cells or platelets) flowing into a reservoir. Filters may also be present on the lines of one or more containers used for addition of materials, such as containers 40, 46, 48, 64, 66, 68, and 70 in order to filter or sterilize materials as they enter the fluid circuit. Additionally, a cell counting component may also be added. A spill detection module or device may also be included.

Before beginning a blood processing procedure, if there are any fluid containers that are not integrally formed with the fluid flow circuit 10, they may be connected to the fluid flow circuit 10 (e.g., by piercing a septum of a tube of the fluid flow circuit 12 or via a luer connector), with the fluid flow circuit 10 then being mounted to the blood separation device 20. Additionally, any inlet containers may be filled with the appropriate fluid, such as buffer or solution. An integrity check of the fluid flow circuit 10 may be executed by the controller 16 to ensure the various components are properly connected and functioning.

To begin a blood processing procedure, an operator may select (e.g., using the user interface screen 14, see FIG. 3C) the procedure from among the variety of procedures that the device 10 is capable of performing. The operator may enter a variety of information requested by the system controller that allows the controller to better carry out the procedure. The controller can be provided with the desired cell modification processes, the solutions being use, type of blood cells being modified, total blood volume needed for the process or of the blood source, a WBC pre-count, WBC subset pre-count or the initial WBC concentration of the blood of the blood source, and a WBC post-count or a target platelet concentration to be achieved for the blood of the blood source by the end of the procedure. The total amount of blood to be processed may also be provided to the system controller. Additionally, various patient measurements such as height, weight, etc. may also be added.

When the system controller has received all of the necessary input, performed the necessary preliminary calculations and status checks (e.g., to confirm that the flow circuit 10 is properly installed and that the various components of the system 21 are functioning properly), the blood source is connected to the fluid flow circuit 10 (e.g., by phlebotomizing a donor or attaching a whole blood container), and a blood processing procedure may begin. The blood source may include a donor or a container of blood or other suspension of cells. The blood or other cellular starting material may be whole blood, blood components obtained by, for example, apheresis, or other nucleated cell suspensions.

Blood is introduced into the system by either an active or passive user adjacent portion of the fluid flow circuit 10 or by connection of a blood or blood component container. The blood flows from the donor into the main line L1 and reservoir 40. In an initial stage, which is referred to herein as a “blood prime” stage and shown in FIG. 9, selected components of the fluid flow circuit 10 are primed using blood 41 from a blood source, particularly being stored in reservoir 40. It is also within the scope of the present disclosure for the fluid flow circuit 10 to be primed using a different priming fluid, such as saline.

During the blood prime stage, whole blood is drawn into the fluid flow circuit 10 from the reservoir via line L2. The blood travels through L1 to the patient adjacent portion of the fluid flow circuit 10. Although not shown in FIG. 9, the priming process may be done to other components of the fluid flow circuit, such as parts of the processing portion 25 of the fluid flow circuit 10.

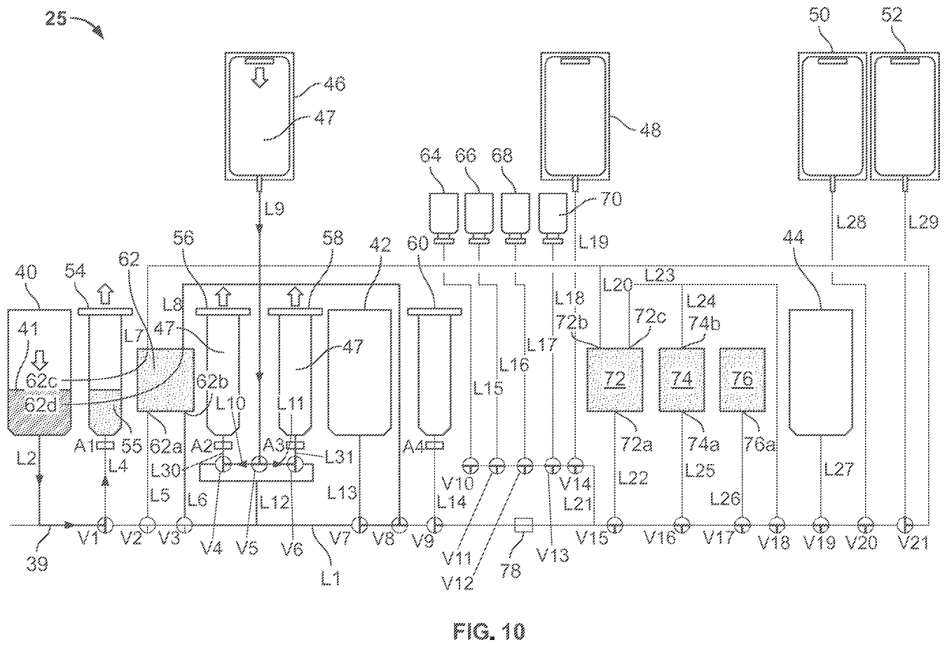

The separation stage may then be initiated. In the first step of the stage, shown in FIG. 10, blood 41 loaded into pump 54 and buffer 47 is loaded into pumps 56 and 58. Blood or blood components 41 from Reservoir 40 is/are pulled or loaded into Pump 54 by passing through lines L2, L1, valve V1 and line L4. Buffer 47 from container 46 is pulled into Pumps 56 and 58. Specifically, the buffer passed from container 46 through line L9 and valve V5 to either line L10, through valve V4 and line L30 to pump 56 or line L11 through valve V6 and line L31 to pump 58.

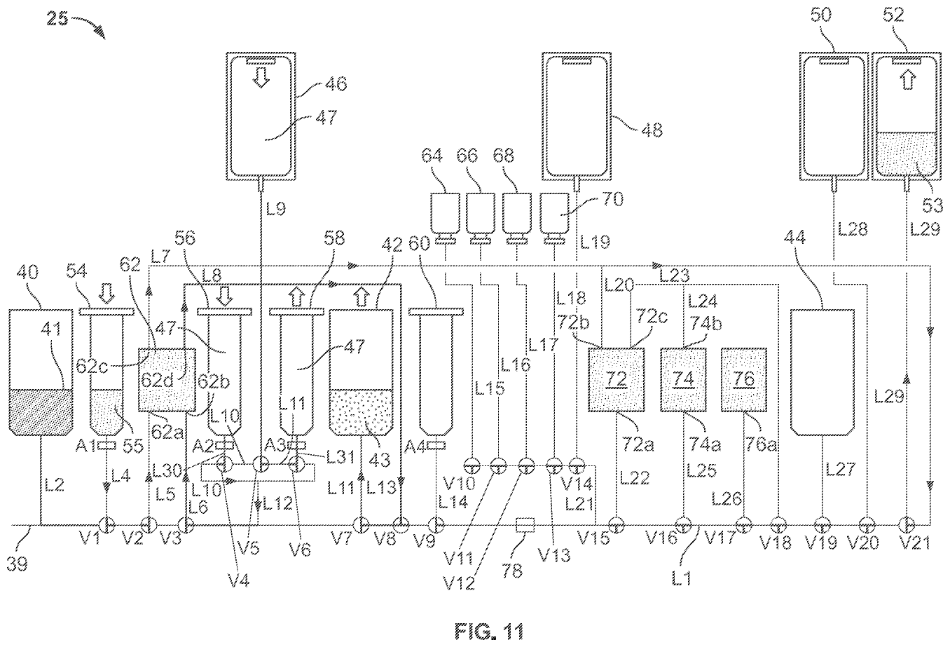

Buffer 47 may optionally be also directed to the pump 54. In a second step of the separation stage, shown in FIG. 11, the pumps 56 and 58 are pressurized to operating pressure and flow into the separator module 62. Pumps 56 and 58 may be sequenced in order to provide continuous flow. Buffer 47 is pushed from either pump 56 or pump 58 through line L12, valve V3 and line L6 to the separation module 62 at inlet 62b. Pump 54 also pushes blood components 55 through line L4, valve V1, valve V2 and line L5 into separation module 62 at inlet 62a. The cells are separated based on size. Larger cells/particles 43, such as white blood cells, are separated and directed into reservoir 42 through outlet 62d, by line L8 through valves V8 and V7 to line L13. Small cells/particles 53, such as red blood cells and platelets are directed to the waste container 52 by traveling out of outlet 62c to line L7, line L1, valve V21 and line 29 to waste container 52. The separation module may perform differently depending on the desired separation and particular cell to be modified.

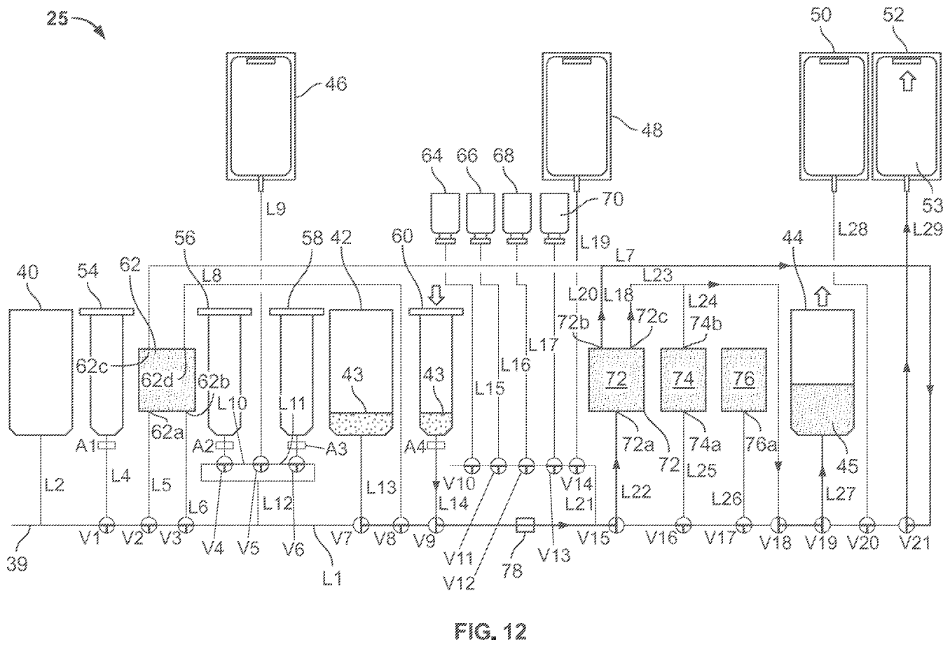

A cell concentration procedure, shown in FIG. 12, may then be performed. Large cell material 43 is loaded or pulled into pump 60 from reservoir 42 by passing through line L13, valves V7, V8, and V9 to line L14. Pump 60 is then pressurized to operating pressure and the large cell material is pushed through line L14 and valve V9 to line L1 and then through valve V15 and line L22 to the inlet 72a of the concentrator module 72. The concentrator module produces concentrated cells 45 and supernatant 59. Supernatant 59 is directed towards the waste container 52 by passing through outlet 72b to line L20 to line L7, through valve 21 and to line L29. Concentrated cells 45 are directed towards reservoir 44 by passing out of outlet 72c to line L23, through valve 18, line L1 and valve V19 to line L27. Concentrated cells may be subsequently diluted, then optionally reconcentrated by drawing again into pump 60 from reservoir 44 and repeating the concentration module 72 until cells are at a target concentration. Cell concentration may be sensed during concentration or transfer states.

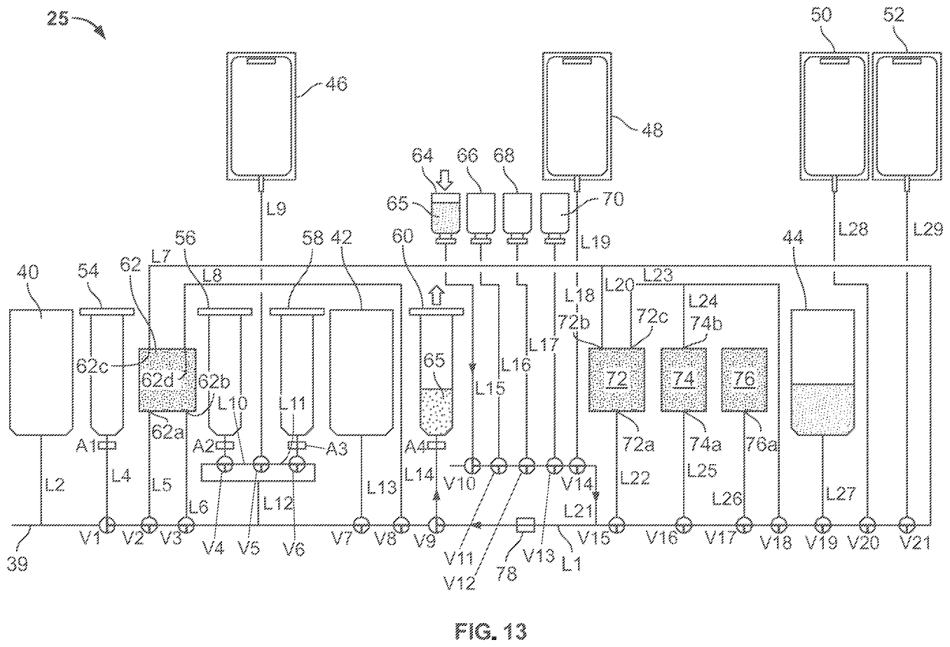

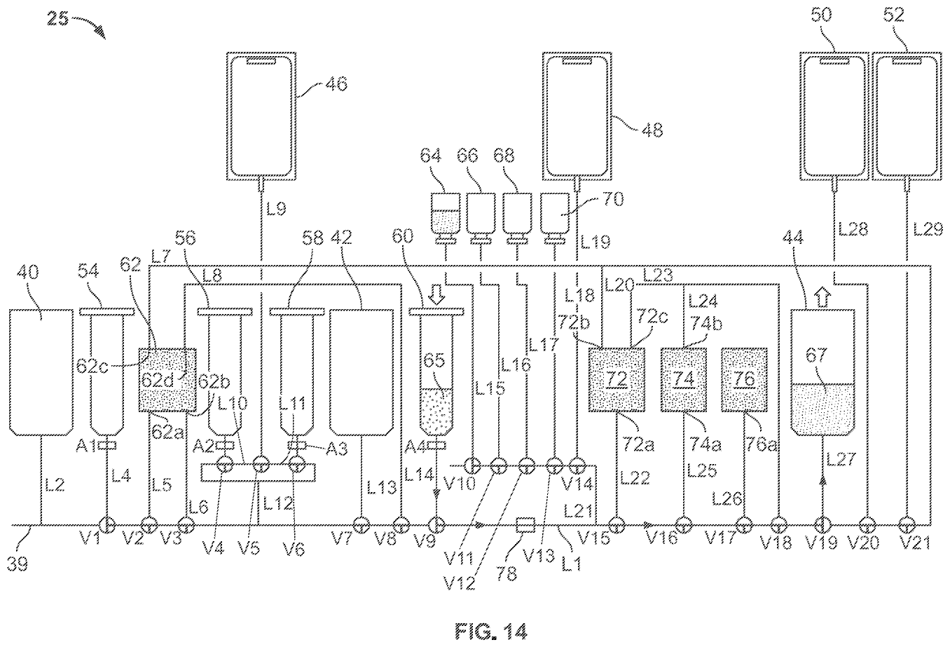

A cell formulation stage or procedure may also be performed. This may be done to concentrated cells 45 (as shown in FIGS. 13 and 14) or on the separated cell material (such as large cell material 43). Concentrated cells 45 or large cell material 43 may be treated with at least one solution, buffer, or combinations of solution(s) and buffer(s) in reservoirs 42, 44. In an exemplary method, a first step, shown in FIG. 13, includes a measured volume of solution 65 drawn into pump 60. The solution 65 from container 64 travels through line L15, valves 10, 11, 12, 13, 14 to line L21 and L1 to valve 9 and line L14. In a second step of the cell formulation stage of the exemplary method, shown in FIG. 14, solution 65 is pumped toward Reservoir 44, which may include concentrated cells 45. Specifically, the solution travels through line L14 and valve v9 to line L1, through valves v15, v16, v17,v18, and v19 to line L27. The cells combine with the solution and may be incubated into suspension 67. During hold or incubation steps, the suspension may be drawn back and forth between reservoir 44 and pump 60 to prevent settling. In an alternative second step of the cell formulation stage (when added to large cell material 43), solution 65 may be instead pumped toward reservoir 42, where the large cell material 43 is held after the separation stage, shown in FIG. 11. Specifically, the solution travels through line L14 and valve v9 to line L1, through valves v8 and v7 to line L13. The cells combine with the solution and may be incubated in reservoir 42. During the incubation, the suspension may be drawn back and forth between reservoir 42 and pump 60 to prevent settling.

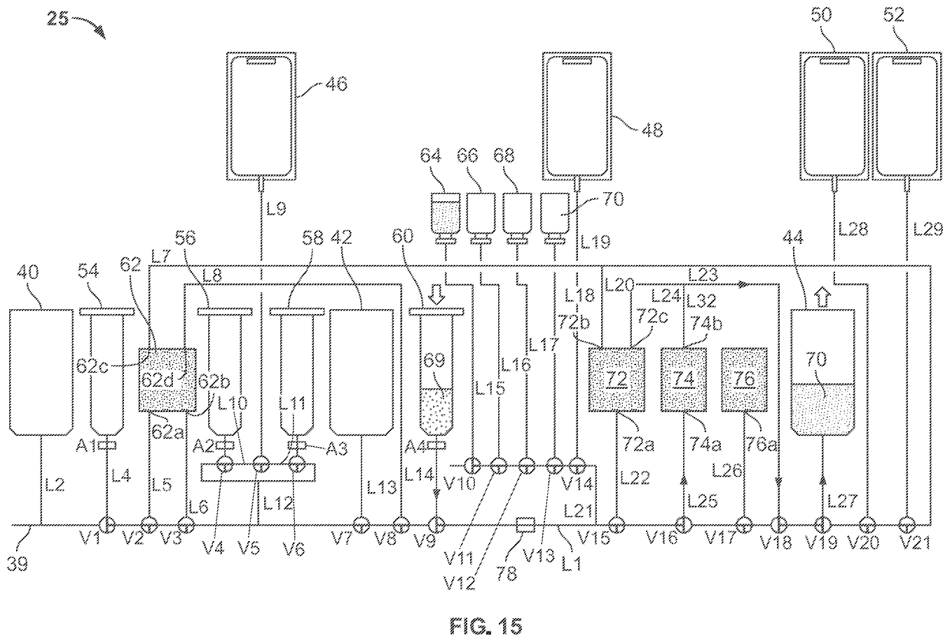

A gene delivery stage procedure may also be performed, as shown in FIG. 15. Cells 69 which have been formulated with a gene modification solution can be directed from Pump 60 to the Gene Delivery Module 74 in which payload is administered to the cell. Similar to the process shown in FIGS. 13 and 14, cells 69 may be formed by first pulling a gene modification solution into pump 60 and pushing the solution into one of reservoir 42 or 44, which include blood component cells. These cells 69 can be formed from the large cell material 43 or cells which have already been modified, such as by the cell concentrating or formulating stage. The cells 69 may then be pulled into pump 60. As shown in FIG. 15, the formulated cells pass from pump 60 through line L14, valve v9 to line L1, through valves v15 and v16 to L25 and into gene delivery module 74 through an inlet 74a. This module 74 may leverage electroporation, mechanoporation, or other flow-through transfection methods to introduce the payload. The modified cells 70 exit the module 74 at outlet 74b to line L32 and line L23 through valves v18 and v19 to line L27.

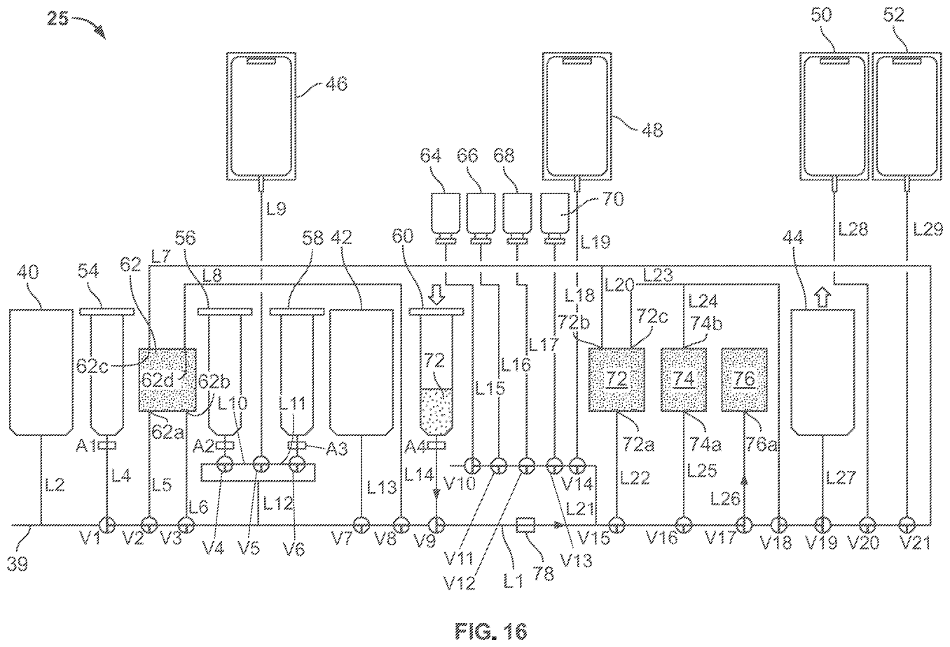

A gene delivery stage procedure may also be accomplished without passing through the Gene Delivery Module 74. Instead, a gene solution is simply added in the above-described cell formulation stage (to large cell material 43). For instance, lipid nanoparticles may incubate with the cells in order for them to introduce their payload. Optionally, a cell selection stage procedure may also occur or be performed. Cells 72 which have been formulated with antibodies, beads, or other solutions that can identify cells based off surface markers or phenotype can be passed into a selection chamber 76. Similar to the process shown in FIGS. 13 and 14, cells 72 may be formed first by pulling the cell identifying solution into pump 60 and pushing the solution into one of reservoir 42 or 44, which include blood component cells. These cells 72 can be formed from the large cell material 43 or cells which have already been modified, such as by the cell concentrating stage, formulating stage, or gene delivery stage. The cells 72 may then be pulled into pump 60. As shown in FIG. 16, the cells 72 pass from pump 60 through line L14, valve v9 to line L1, through valves v15, v16, and v17 to L26 and into the cell selection module 76 through an inlet 76a. Cells can be positively or negatively isolated and the target cell fractions can be directed towards an appropriate reservoir or output container.

Although possible cell modification stages have been described, it is not required that each stage be performed on the collected blood cells in any given blood processing procedure, but may depend on the resulting cell make-up requirements. The cells may be modified in at least one stage and can be modified in all stages, or a combination of some of the stages. The stages do not have to be performed in a specified order and can each be done multiple times. Most importantly, the cells are separated and modified in the same procedure and system.

Once the cells are modified in at least one of the described cell modifying modules, these cells may be directed to the dose/sample container 50 (FIG. 8) or optionally returned to the donor/patient through the fluid flow circuit. If directed to container 50, the container may be removed from the circuit. The modified cells may pass back through to the donor/patient by directing the cells back through line L1 of the processing portion 25 of the fluid flow circuit 10 to the patient adjacent portion 22 of the fluid flow circuit. Various pumps and valves may be utilized to direct the fluid back to the patient. The patient adjacent portion 22 of the fluid flow circuit may initiate and direct the fluid flow back to the patient.

There are a few factors that may be used to determine whether the cells are collected in a dose or reinfused back to the patient. These include guidelines imposed by regulators (like the FDA), the duration of processing that is required and whether patient connection for the duration is acceptable (if the processing take hours, perhaps the patient need not be connected during the duration of processing, the need for extensive release testing prior to reinfusion, or if the cell require additional handling (such as culture/expansion, offline dosing, etc.).

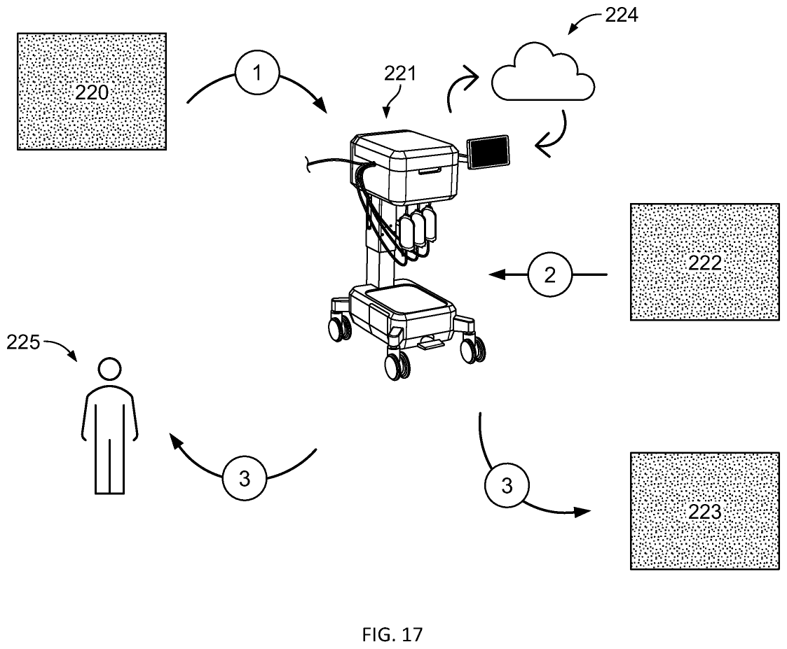

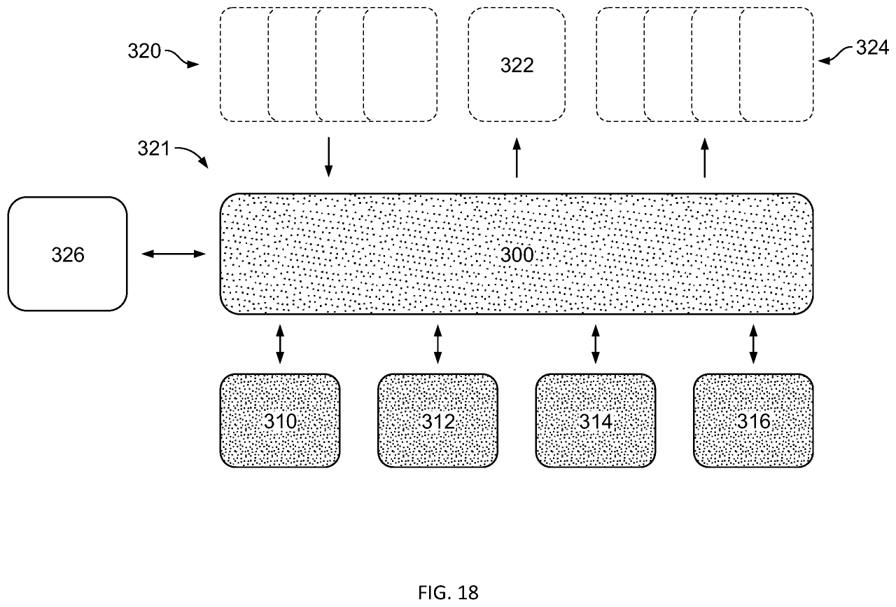

A more particular embodiment of the systems and methods described above are depicted in FIGS. 17-22 and will now be described. It will be understood that such systems and methods may utilize many of the aspects and features described previously to provide a single, modular system configured for point-of-care i.e., bedside use. For example, device 221 shown in FIG. 17 may be the mobile modular device 21 of FIGS. 2, 3B and 3C. FIG. 17 shows biological cell processing system 221 including the device 20 and the fluid flow circuit 10 of the current disclosure and a possible procedure indicated 1-3. The blood processing system receives blood such as whole blood, blood components which may typically be obtained by apheresis, or other nucleated cell suspensions 220 from a source at step 1, wherein the source may be a donor or blood bag of previously collected blood or blood component. The system uses protocols or procedures from internal or external data sources 224 and performs various processes in the system. The system can also share generated data (at this step or a further step) with external sources 224. Data may be instrument statuses, error logs, procedure data/records, or live sensor data. At step 2, additional fluids or agents, such as additives or solutions 222 are added to the system, through at least one module. There are two alternate step 3's in which the final product can be returned to a patient 225 or collected in a bag or other output receiver 223. This entire process may take place in less than a day, less than 12 hours, or even under 6 hours.

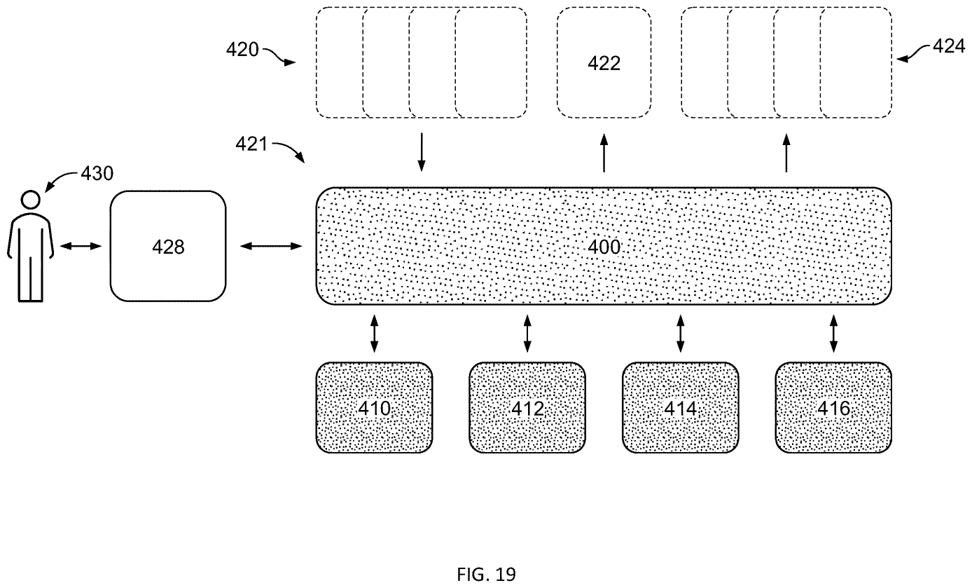

FIGS. 18 and 19 show two block diagrams of the blood processing system, with the difference being the type of blood source utilized. System 321 which includes as its source a bag or container with previously collected blood, blood components obtained by apheresis, or other nucleated cell suspensions 326 (obtained, for example, by apheresis) and System 421 utilizes a donor 430 and includes a donor management module 428. Donor management module can be either of the passive or active user adjacent first portions discussed above and shown in FIGS. 6 and 7. The systems 321, 421 each include a fluid handling and control system 300, 400, as a central and holding component for the other modules and inputs/outputs. A plurality of different modules may be added or removed from the system (such that only one, two, or three modules may also be present include of all four). The modules may include a microfluidic sorter 310, 410, a microfluidic concentrator 312,412, and optionally a cell selection module 314, 414, and a “cargo delivery” module 316, 416. Inputs to the system can include processing buffers and solutions 320, 420. Outputs of the system can include waste material 322, 422 and samples or outputs 324, 424.

Although the systems 221, 321, and 421 utilize microfluidic portions, the system may also allow for processing of larger amounts of whole blood or blood components obtained from whole blood. The flow rates through the system may be from 5 mL/min to 70 mL/min. The systems 221, 321, and 421 as a whole can process up to 500 mL of whole blood, with the withdrawal or draining comprising up to an hour from the blood source.

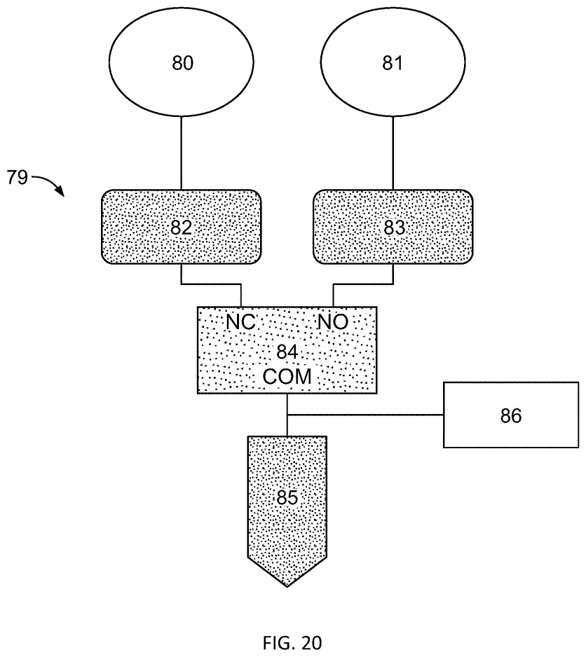

Systems 221, 321, and 421 may utilize pneumatic syringe pumps as described above for delivering starting cellular material, buffers or other agents and additives to the microfluidic sorter and concentrator. The system may further include a pressure control system 79, shown in FIG. 20. The system of syringe pump 85 includes a pressure tank 80 and a vacuum tank 81, with associated pressure regulator 82 and vacuum regulator 83. The three-way valve 84 includes connections that are or normally open (NO), or normally closed (NC), and for common (COM).

The system further includes a two-way normally open valve 86 which can function as a vent. These tanks are maintained at 90-110 psi and <-10 psi. Each syringe has a syringe controller which has a regulator to set syringe pressure.

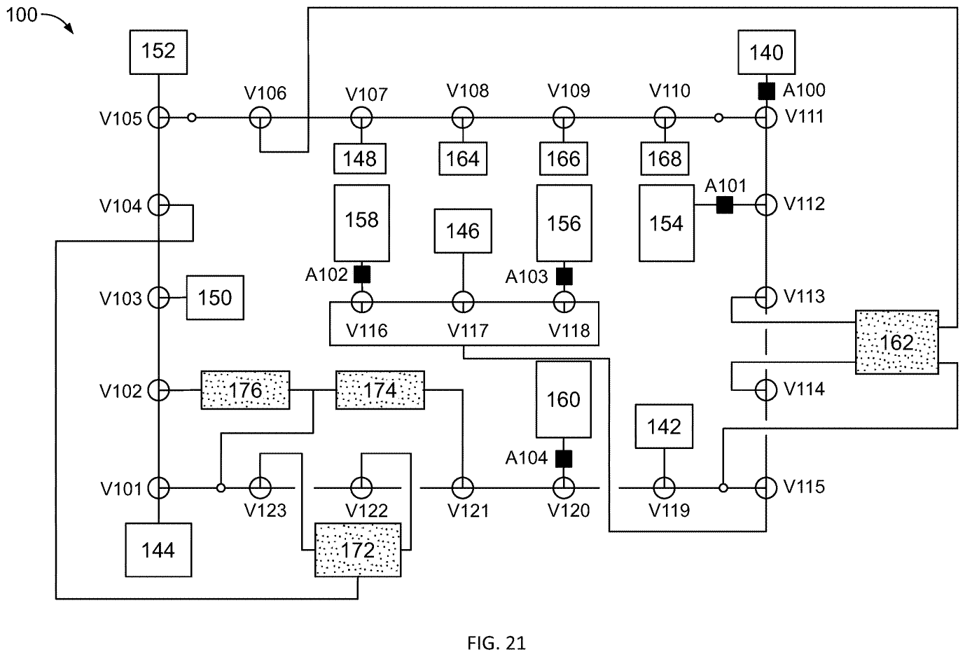

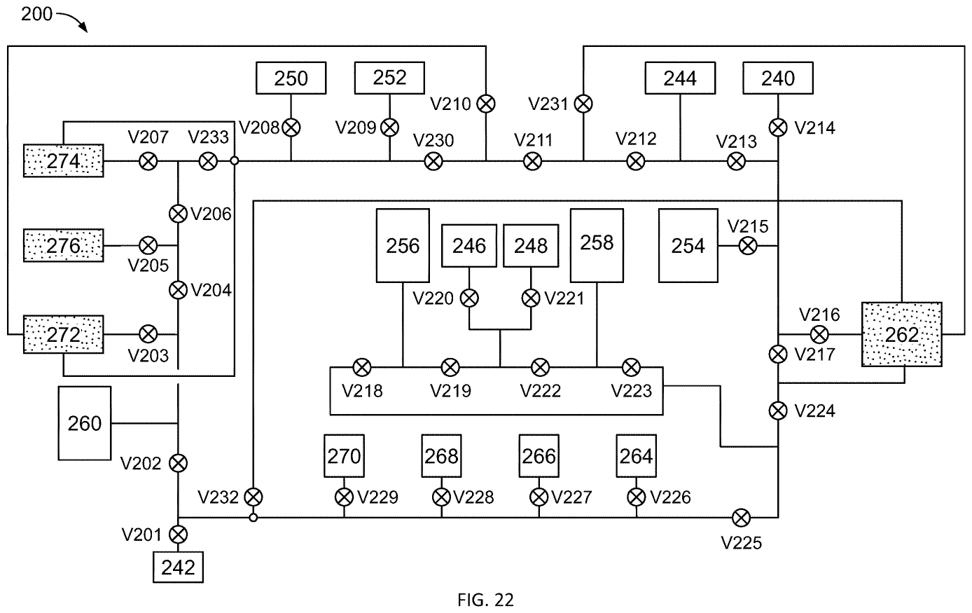

The valve system of the current embodiment of the blood processing system 221, 321, 421 can include stopcock valves or solenoid valves. Fluid flow circuits 100 and 200 in FIGS. 21 and 22 show fluid flow paths with stopcock valves (FIG. 21) and solenoid valves (FIG. 22), respectively.

More particularly, FIG. 21 shows a fluid flow circuit 100 of a blood processing system of the current embodiment. Valves V101-V123 can be any known type of stopcock valve. The fluid flow circuit 100 connects and established fluid communication between and among component parts, similar to those described above including: first buffer container 146, second buffer container 148, reservoirs 140, 142, and 144, sample/dose container 150, waste container 152, pumps 154, 156, 158, and 160, solution or liquid containers 164, 166, and 168. The fluid flow circuit may also include air detectors A100-A104. The fluid flow circuit 100 further includes a microfluidic sorter module 162, a cell concentrator module 172, and optionally a cargo delivery module 174, and a cell selection module 176.

FIG. 22 shows a fluid flow circuit 200 of a blood processing system of the current embodiment. Valves V201-V233 can be any known type of solenoid valve. The valves may be part of a solenoid driven cassette, of the type shown in U.S. Patent Publication No. 2017/0290972, filed Mar. 29, 2017, which is incorporated by reference. The fluid flow circuit 100 connects and established fluid communication between and among component parts, similar to those described above including: first buffer container 246, second buffer container 248, reservoirs 240, 242, and 244, sample/dose container 250, waste container 252, pumps 254, 256, 258, and 260, solution or liquid containers 264, 266, and 268. The fluid flow circuit 200 may also include air detectors, although not specifically illustrated. The fluid flow circuit 200 further includes a microfluidic sorter module 262, a cell concentrator module 272, and optionally a cargo delivery module 274, and a cell selection module 276.

The microfluidic sorter module 162, 262 may have properties that are common with the above-described separation module, such as shown in FIG. 8 in the schematic of the fluid flow circuit 25. This microfluidic sorter module 162, 262 may be a substantially or completely passive separation operation, without reliance on moving parts or complex systems, such as that in a centrifugal separation or spinning membrane separation. In an embodiment, the microfluidic sorter module may be a chip or cartridge. The chip may have multiple layers including an interface layer, which enables fluidic connections to other components, such as tubing. The chip may also have a lid layer for sealing the lower fluidic layer. The fluidic layer includes the microfluidic channels and carries out the cell sorting and concentration. The microfluidic chip or cartridge may operate to sort the cells based on inertia. If whole blood is introduced to the microfluidic sorter module dilution is required. Apheresis product does not require the same dilution. Microfluidic chips or cartridges utilized in the microfluidic sorter module can be of the type described in U.S. Pat. No. 10,150,116.

The cell concentrator module 172, 272 may include the properties described above in relation to the concentrating module, such as shown such as shown in FIG. 8 in the schematic of the fluid flow circuit 25. The cell concentrator module 172, 272 may include a number of concentrator channels. In one embodiment, the concentrator includes at least 200 channels, and in another embodiment includes at least 250 concentrator channels. The concentrator may operate passively, without any moving parts or complex control systems. The concentrator module may operate at greater than 100 mL/min. The cell concentrator may operate to concentrate at least one cellular component by 10×. In one example, the microfluidic sorter module separates nucleated cells from anucleated cells. These nucleated cells can then be concentrated in the cell concentrator module. The cell concentrator module may include the cell concentrator technology of the type described in U.S. Pat. No. 10,150,116. The microfluidic sorter module and cell concentrator module may be connected and added and removed from the system as one piece or they may each be separate pieces.

The fluid flow circuit may include a cargo delivery module 174, 274 or feature which utilizes microfluidic mechanoporation in order to accomplish intracellular cargo (for example, gene) delivery. Cells and target material in suspension undergo high speed cell deformation which causes a temporary disruption of the cell membrane and allows the target material to enter the cell. Afterward, the membrane reseals. The cargo delivery module 174, 274 may include delivery materials such as mRNA, siRNA, saRNA, polymers, proteins and peptides, antibodies, viruses, labelling molecules, small molecules, and CRISPR RNPs. The validated cell types may include peripheral blood mononuclear cells, t cells, b cells, monocytes, natural killer (NK) cells, hematopoietic stem cells, induced pluripotent stem cells, and red blood cells.

The fluid flow circuit may include a cell selection module 176, 276 which may be column-based and have immunophenotypic cell selection. In an embodiment, the cell selection module 176, 276 may include a non-magnetic affinity-chromatography based cell selection column that operates to isolate a target cell (such as CD3+) from a population including non-target cells. The process uses a polymer matrix with a ligand and ligand binding partner which binds the target antigen (target cell, CD3+). A competing molecule is then added to release the target cell. The resulting target cells may require a buffer rinse to be label-free. The cell selection technology utilized can be that described in Next Generation Automated Traceless Cell Chromatography Platform for GMP-compliant Cell Isolation and Activation. Scientific Reports (2022) 12:6572.

Any components not specifically described in relation to the blood processing system 221, 321, 421 are the same or similar to those described above in relation to the blood processing system 21. The blood processing systems 221, 321, 421 include at least a controller and a fluid flow circuit 100, 200. The fluid flow circuit includes at least a microfluidic sorter module, a cell concentrator module, at least one pump, at least one fluid reservoir for holding fluids during a blood processing procedure, a valve system, at least one fluid source container, a blood source access device, and a plurality of conduits fluidly connecting the components of the fluid flow circuit. The fluid flow circuit may optionally include at least one cell modification module, such as a cargo delivery module and a cell selection module. The blood processing system may include further components such as sensors, air detectors, additional pumps, and weigh scales, as described above.

### EXAMPLE

The following collection and processing workflow could be accommodated using combinations of the states and procedures previously described. Using the fluid flow circuit, the system collects a target volume of anticoagulated whole blood from a patient based off target cell peripheral blood pre-counts into reservoir 40. The anticoagulated whole blood is separated in the separation module and white blood cells are isolated in reservoir 42. The white blood cells are then passed through the concentration module 72 and concentrated 25×, which may include multiple passes through the concentration module. A biotinylated antibody additive is introduced into the system and the white blood cells and incubated in reservoir 44 with the biotinylated antibody, which is used to label all cells except for CD3+ cells. Afterward, streptavidin coated magnetic particles are introduced to the system and reservoir 44 and the mixture is incubated. This mixture is then transferred to the selection module 76, wherein the labeled cells are bound in the chamber. The unlabeled, unbound target CD3+ cells are removed from the selection chamber into a reservoir such as reservoir 42 or 44. The cells are then passed into the concentration module 72 and the isolated CD3+ cells are 10× concentrated. A gene modification solution (e.g., mRNA, CRISPR-Cas9, Transposon/Transposase) is introduced into the system and into the holding reservoir with the CD3+ cells. The cells are then transferred through the gene delivery module 74 and into another reservoir. These cells then can be washed in the separation module 62 with buffer. The cells are then passed through the cell concentration module 72 and concentrated to a target reinfusion target. These concentrated cells are then transferred to a dose container or reinfused into the patient.

Thus, an improved method and system have been disclosed for the processing of blood components. Advantageously, the modular fluid flow circuit described herein includes cell separation and cell modifying components and can produce separated and modified cells in a single modular system, in one procedure. The description provided above is intended for illustrative purposes only and is not intended to limit the scope of the invention to any specific method, system, or apparatus, or device described herein except as may be explicitly delineated above.

### Aspects

Aspect 1. A fluid flow circuit for use in a blood processing system comprising: a microfluidic sorter module; a cell concentrator module; at least one pump; at least one fluid reservoir for holding fluids during a blood processing procedure; a valve system; at least one fluid source container; a blood source access device; and a plurality of conduits fluidly connecting the components of the fluid flow circuit.

Aspect 2. The fluid flow circuit of Aspect 1, wherein the fluid flow circuit further comprises at least one cell modification module.

Aspect 3. The fluid flow circuit of Aspect 2, wherein the at least one cell modification module includes a cell selection module.

Aspect 4. The fluid flow circuit of any of the preceding Aspects, wherein the at least one fluid source container is a buffer container.

Aspect 5. The fluid flow circuit of any of the preceding Aspects, wherein the at least one fluid source container is a solution container.

Aspect 6. The fluid flow circuit of any of the preceding Aspects, wherein the microfluidic sorter module is configured to separate blood components based on size.

Aspect 7. The fluid flow circuit of any of the preceding Aspects, wherein the least one pump includes a plurality of pneumatic syringe pumps.

Aspect 8. A blood processing system comprising: the fluid flow circuit of any of the preceding Aspects; and a reusable hardware device comprising a controller configured and/or programmed to initiate and at least partially control the processing of biological cells through the fluid flow circuit.

Aspect 9. The blood processing system of Aspect 8, wherein the blood processing system is a modular system.

Aspect 10. The blood processing system of Aspect 8, wherein the controller is configured to operate the at least one pump and the valve system to convey biological cells from a source access device through the fluid flow circuit; execute sorting of the blood into two or more cellular components using the sorting module; and concentrating at least one cellular component of the blood with the cell concentrator module to produce at least one concentrated cellular component.

Aspect 11. The blood processing system of Aspect 10, wherein the controller is further configured to operate the at least one pump and the valve system to convey a solution to the at least one cellular component.

Aspect 12. The blood processing system of Aspect 10, wherein the controller is configured to operate the at least one pump and the valve system to convey at least one concentrated cellular component through a cell modification module.

Aspect 13. The blood processing system of Aspect 12, wherein the controller is configured to execute a modification of the at least one cellular component of the blood by operating the at least one pump and the valve system to convey at least one concentrated cellular component through a gene delivery module.

Aspect 14. The blood processing system of Aspect 12, wherein the controller is configured to execute a modification of the at least one concentrated cellular component of the blood by operating the at least one pump and the valve system to convey at least one concentrated cellular component through a cell selection module.

Aspect 15. The blood processing system of Aspect 10, wherein the microfluidic sorter module is configured to sort blood into two or more components based on sizes of said two or more components.

Aspect 16. The blood processing system of Aspect 15, wherein the size is diameter of the cell.

Aspect 17. The blood processing system of Aspect 10, wherein the cellular components include at least one of white blood cells, red blood cells, and platelets.

Aspect 18. The blood processing system of Aspect 10, wherein the controller is further configured to operate at least one pump and the valve system to collect the at least one concentrated cellular component in a container.

Aspect 19. The blood processing system of Aspect 10, wherein the blood from the blood source access device is removed directly from a patient.

Aspect 20. The blood processing system of Aspect 19, wherein the controller is further configured to operate the at least one pump and valve system to initiate reinfusion of the at least one concentrated cellular component into the patient.

1. A fluid flow circuit for use in a blood processing system comprising:
a microfluidic sorter module;
a cell concentrator module;
at least one pump;
at least one fluid reservoir for holding fluids during a blood processing procedure;
a valve system;
at least one fluid source container;
a blood source access device; and
a plurality of conduits fluidly connecting the components of the fluid flow circuit.

2. The fluid flow circuit of claim 1, wherein the fluid flow circuit further comprises at least one cell modification module.

3. The fluid flow circuit of claim 2, wherein the at least one cell modification module includes a cell selection module.

4. The fluid flow circuit of claim 1, wherein the at least one fluid source container is a buffer container.

5. The fluid flow circuit of claim 1, wherein the at least one fluid source container is a solution container.

6. The fluid flow circuit of claim 1, wherein the microfluidic sorter module is configured to separate blood components based on size.

7. The fluid flow circuit of claim 1, wherein the least one pump includes a plurality of pneumatic syringe pumps.

8. A blood processing system comprising:
the fluid flow circuit of any of the preceding claims; and
a reusable hardware device comprising a controller configured and/or programmed to initiate and at least partially control the processing of biological cells through the fluid flow circuit.

9. The blood processing system of claim 8, wherein the blood processing system is a modular system.

10. The blood processing system of claim 8, wherein the controller is configured to operate the at least one pump and the valve system to convey biological cells from a source access device through the fluid flow circuit;
execute sorting of the blood into two or more cellular components using the sorting module; and
concentrating at least one cellular component of the blood with the cell concentrator module to produce at least one concentrated cellular component.

11. The blood processing system of claim 10, wherein the controller is further configured to operate the at least one pump and the valve system to convey a solution to the at least one cellular component.

12. The blood processing system of claim 10, wherein the controller is configured to operate the at least one pump and the valve system to convey at least one concentrated cellular component through a cell modification module.

13. The blood processing system of claim 12, wherein the controller is configured to execute a modification of the at least one cellular component of the blood by operating the at least one pump and the valve system to convey at least one concentrated cellular component through a gene delivery module.

14. The blood processing system of claim 12, wherein the controller is configured to execute a modification of the at least one concentrated cellular component of the blood by operating the at least one pump and the valve system to convey at least one concentrated cellular component through a cell selection module.

15. The blood processing system of claim 10, wherein the microfluidic sorter module is configured to sort blood into two or more components based on sizes of said two or more components.

16. The blood processing system of claim 15, wherein the size is diameter of the cell.

17. The blood processing system of claim 10, wherein the cellular components include at least one of white blood cells, red blood cells, and platelets.

18. The blood processing system of claim 10, wherein the controller is further configured to operate at least one pump and the valve system to collect the at least one concentrated cellular component in a container.

19. The blood processing system of claim 10, wherein the blood from the blood source access device is removed directly from a patient.

20. The blood processing system of claim 19, wherein the controller is further configured to operate the at least one pump and valve system to initiate reinfusion of the at least one concentrated cellular component into the patient.
